(19) United States
(12) Patent Application Publication
(10) Pub. No.: US 2014/0142153 A1
(43) Pub. Date: May 22, 2014

(54) BENDAMUSTINE FORMULATIONS

(71) Applicants: Chandrasekhar Kocherlakota, Secunderabad (IN); Tarun Singh, District Binjor (IN); Nagaraju Banda, Hyderabad (IN); Nirmal Khati, District Nainital (IN); Sachin Sharma, District Kangra (IN); Prasad Vure, Secunderabad (IN); Aparna Mulupuru, Hyderabad (IN); Navin Vaya, Kota (IN); Amit Anil Charkha, Wardha (IN)

(72) Inventors: Chandrasekhar Kocherlakota, Secunderabad (IN); Tarun Singh, District Binjor (IN); Nagaraju Banda, Hyderabad (IN); Nirmal Khati, District Nainital (IN); Sachin Sharma, District Kangra (IN); Prasad Vure, Secunderabad (IN); Aparna Mulupuru, Hyderabad (IN); Navin Vaya, Kota (IN); Amit Anil Charkha, Wardha (IN)

(73) Assignees: DR. REDDY'S LABORATORIES, INC., Bridgewater, NJ (IN); DR. REDDY'S LABORATORIES LTD., Hyderabad, AP (IN)

(21) Appl. No.: 13/981,262

(22) PCT Filed: Jan. 25, 2012
(86) PCT No.: PCT/US2012/022561

§ 371 Date: Dec. 3, 2013

Related U.S. Application Data
(60) Provisional application No. 61/453,796, filed on Mar. 17, 2011.

(30) Foreign Application Priority Data
Jan. 25, 2011 (IN) 241/CHE/2011

(51) Int. Cl. A61K 31/4184

(52) U.S. Cl. CPC A61K 31/4184
USPC 514/394; 548/310.1

(57) ABSTRACT

Aspects of the present appliction relate to pharmaceutical formulations comprising bendamustine or its pharmaceutically acceptable salts, isomers, racemates, enantiomers, hydrates, solvates, metabolites, polymorphs, and mixtures therof, suitable for phamaceutical use. Aspects further provide methods of producing stable bendamustine compositions.

### INTRODUCTION

Aspects of the present application relate to pharmaceutical formulations comprising bendamustine or its pharmaceutically acceptable salts, isomers, racemates, enantiomers, hydrates, solvates, metabolites, polymorphs, and mixtures thereof suitable for pharmaceutical use. Aspects further provide methods of producing stable bendamustine compositions.

The drug having the adopted name “bendamustine” has chemical names: (4-{5-[bis(2-chloroethyl)amino]-1-methyl-2-benzimidazolyl}butyric acid; or 1H-benzimidazole-2-butanoic acid, 5-[bis(2-chloroethyl)amino]-1-methyl-; and is an atypical structure with a benzimidazole ring, the structure including an active nitrogen mustard. Bendamustine has an empirical molecular formula C₁₆H₂₁C₁₂N₃O₂, a molecular weight of 358.3, and structural Formula I.

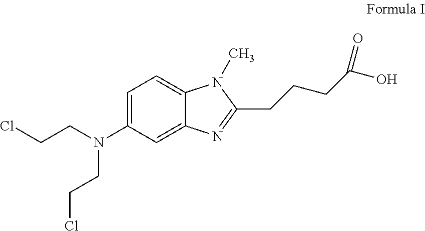 [SMILES: Cn1c(CCCC(=O)O)nc2cc(N(CCCl)CCCl)ccc21]

The salt bendamustine hydrochloride is an alkylating agent, originally synthesized in 1963 at the Institute for Microbiology & Experimental Therapy in Jena, German Democratic Republic, with the intent to produce an agent with both alkylating and antimetabolite properties. Jenapharm (now Schering AG) formerly marketed it in Germany under the trade name Cytostasan from 1971 to 1992. Cytostasan was a lyophilised powder for solution for injection (vials) conatining 25 mg of Bendamustine HCl. It was widely used but never studied systematically in patients until the 1990s, then German investigators demonstrated its clinical activity in a number of malignancies. Since 1993, Ribosepharm was marketing bendamustine in Germany under the brand name Ribomustin RBO. Ribomustin is available as a lyophilized powder for injection, containing 100 mg of drug in each 50 mL vial, or 25 mg of drug in each 20 mL vial, also comprising mannitol, and indicated for the treatment of chronic lymphocytic leukemia. The lyophilized powder is reconstituted as close to the time of patient administration as possible with 40 mL (for a 100 mg product) or 10 mL (for a 25 mg product) of sterile water for injection. The reconstituted product then is further diluted to 500 mL with 0.9% sodium chloride for injection. The route of administration is by intravenous infusion over 30 to 60 minutes.

Another bendamustine product is sold in the United States by Cephalon, Inc. as TREANDA® for Injection, a lyophilized powder in a single-use vial indicated for the treatment of patients with chronic lymphocytic leukemia and indolent B-cell non-Hodgkin's lymphoma. A 25 mg dose vial contains 25 mg of bendamustine hydrochloride and 42.5 mg of mannitol, and a 100 mg dose vial contains 100 mg of bendamustine hydrochloride and 170 mg of mannitol. TREANDA is intended for intravenous infusion only after reconstitution with Sterile Water for Injection USP, and then further dilution with either 0.9% Sodium Chloride Inj.ection, USP, or 2.5% Dextrose/0.45% Sodium Chloride Inj.ection, USP. The pH of the reconstituted solution is 2.5-3.5. TREANDA is supplied as a sterile non-pyrogenic white to off-white lyophilized powder, in a single-use vial.

Bendamustine hydrochloride is very unstable in an aqueous solution. The bis-2-chlorethylamino bond is hydrolyzed in weak acid, neutral, or alkaline solution. Monohydroxybendamustine [HP-1] is formed rapidly in the presence of water. Bendamustine ethyl ester [BM1EE] is formed when bendamustine reacts with ethyl alcohol. BM1EE can be formed during drug substance manufacturing, e.g., during recrystalization and/or purification processes. BM1EE is a more potent cytotoxic drug than bendamustine.

Due to its degradation in aqueous solutions (similar to other nitrogen mustards), bendamustine is provided in lyophilized products. The finished lyophilizate is unstable when exposed to light. Therefore, the product frequently is stored in brown or amber-colored glass bottles. The lyophilized formulation of bendamustine contains degradation products that may occur during manufacturing of the drug substance and/or during the lyophilization process to make the finished drug product. Moreover, reconstitution of the lyophilized powder is difficult and the reconstitution time depends on the solvent used during lyophilisation and the manufacturing parameters. Reports from clinical experience indicate that reconstitution can require at least fifteen minutes and may require as long as thirty minutes. In addition to being troublesome and time-consuming for the healthcare professional responsible for reconstituting the product, the lengthy exposure of bendamustine to water during the reconstitution process increases the potential for loss of potency and impurity formation, due to hydrolysis of the product by water.

German Democratic Republic (GDR) Patent No. 34727 discloses a method of preparing ω-[5-bis-(beta-chloroethyl)-amino-benzimidazolyl-(2)]-alkane carboxylic acids that are substituted in the 1-position.

GDR Patent No. 80967 discloses an injectable preparation of γ-[1-methyl-5-bis-(beta-chloroethyl)-amino-benzimaidazolyl-(2)-]-butric acid hydrochloride.

GDR Patent No. 159877 discloses a method for preparing 4-[1-methyl-5-bis(2-chloroethyl)amino-benzimidazolyl-2)-butyric acid.

GDR Patent No. 159289 discloses an injectable solution of bendamustine.

An article by N. Ni et al., “Use of Pure t-Butanol as a Solvent for Freeze-Drying: a Case Study,” International Journal of Pharmaceutics, Vol. 226, Issues 1-2, pages 39-46 (2001), reports that 1-(2-chloroethyl)-3-sarcosinamide-1-nitrosourea was more stable in pure tertiary-butanol (TBA) than in pure acetic acid, dimethylsulfoxide, water, or in TBA-water mixtures.

Lyophilized cyclophoshamide is disclosed in U.S. Pat. Nos. 5,418,223, 5,413,995, 5,268,368, 5,227,374, 5,130,305, 4,659,699, 4,537,883, and 5,066,647.

The lyophilized nitrogen mustard compound ifosfamide is disclosed in International Application Publication No. WO 2003/066027 and U.S. Pat. Nos. 6,613,927, 5,750,131, 5,972,912, 5,227,373, and 5,204,335.

Lyophilized formulations of prostaglandin E-1, made by dissolving PGE-1 in a solution of lactose and tertiary-butyl alcohol, are disclosed in U.S. Pat. No. 5,770,230.

U.S. Patent Application Publication No. 2006/0159713 discloses pharmaceutical formulations of lyophilized bendamustine, suitable for pharmaceutical use, and provides methods of producing lyophilized bendamustine. The pharmaceutical formulations can be used for any disease that is sensitive to treatment with bendamustine, such as neoplastic diseases.

Because of their high reactivity in aqueous solutions, nitrogen mustards are difficult to formulate as pharmaceuticals and are often supplied for administration in a lyophilized form that requires reconstitution, usually in water, by skilled hospital personnel prior to administration. Once in aqueous solution, nitrogen mustards are subject to degradation by hydrolysis; thus, the reconstituted product should be administered to a patient as soon as possible after its reconstitution and reconstitution time should be short enough to reduce degradation.

A lyophilised powder contains bendamustine HCl and suitable bulking agent. One of the suitable bulking agents is mannitol. However, formulation the lyophilized powders is difficult. Organic solvents were found to be more suitable to avoid degradation of bendamustine HCl in pre-lyophilization bulk solutions. However, mannitol cannot be dissolved in completely organic solvent systems. Therefore, water should be a part of solvent system for preparing bulk solution. To lower the rate of degradation of bendamustine HCl, solvent systems for manufacturing bulk solution, the sequence of addition of ingredients, temperatures, duration of critical steps in lyophilization, and the like can be critical.

There remains a need for stable formulations of bendamustine HCl that are easy to reconstitute and which have improved impurity profiles over the current commercial lyophilized powder formulations of bendamustine, and having more simple or cost-effective methods of preparation.

### SUMMARY

Aspects of the present application relate to pharmaceutical formulations comprising bendamustine or any of its pharmaceutically acceptable salts, isomers, racemates, enantiomers, hydrates, solvates, metabolites, polymorphs, and mixtures thereof suitable for pharmaceutical use. Aspects further provide methods for producing stable bendamustine compositions.

In embodiments, the present application provides lyophilized bendamustine formulations prepared by methods that involve the use of an organic solvent with water in suitable proportions, as pre-lyophilization bulk solvent systems.

In embodiments, the present application provides lyophilized bendamustine formulations prepared by methods that involve the use of an organic solvent with water, as pre-lyophilization bulk solvent systems, wherein such organic solvents do not include tertiary-butanol (TBA) or ethanol.

In embodiments, the present application provides lyophilized bendamustine formulations prepared by methods that involve the use of organic solvents with water, as pre-lyophilization bulk solvent systems, wherein such organic solvents include acetone, acetonitrile, or mixtures thereof.

In embodiments, the present application of provides lyophilized bendamustine formulations comprising a bulking agent, wherein a bulking agent comprises mannitol, lactose, sucrose, or any mixtures of two or more thereof.

In embodiments, the present application includes lyophilized bendamustine formulations having drug-related impurities within commercially acceptable limits, the impurity concentrations being maintained during storage for commercially relevant times.

In embodiments, the present application includes processes for manufacturing lyophilized bendamustine formulations, comprising controlling the concentrations of bendamustine degradants in the final product, such that the concentration of HP1 (shown as Formula II below) is less than about 0.9%, or less than about 0.5%, and, at the time of product expiration, the concentrations of bendamustine degradant compounds are less than about 7%, or less than about 5%, after the product is stored at about 2° C. to about 30° C. Impurity contents herein are expressed as weight percentages of the label bendamustine content.

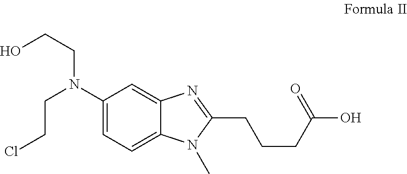 [SMILES: Cn1c(CCCC(=O)O)nc2cc(N(CCO)CCCl)ccc21]

In embodiments, the present application includes bendamustine formulations that comprise an excipient and a stabilizing concentration of an organic solvent. A representative composition includes bendamustine HCl at a concentration of about 22 mg/mL, and mannitol at a concentration of about 37 mg/mL, in a solvent comprising acetone or acetonitrile in the range of 5 to 50 volume percent with water for injection.

In embodiments, the present application includes processes for manufacturing lyophilized bendamustine formulations, which provides minimum reconstitution times.

In embodiments, the present application includes processes for manufacturing lyophilized bendamustine formulations, wherein crystallinity of the active pharmaceutical ingredient is retained in a finished formulation.

### BRIEF DESCRIPTION OF THE DRAWINGS

FIG. 1 shows powder X-ray diffraction (PXRD) patterns of materials from Examples 7A and 7B.

FIG. 2 shows PXRD patterns of materials from Example 7B.

### DETAILED DESCRIPTION

Aspects of the present application relate to pharmaceutical formulations comprising bendamustine or any of its pharmaceutically acceptable salts, isomers, racemates, enantiomers, hydrates, solvates, metabolites, polymorphs, and mixtures thereof suitable for pharmaceutical use. Aspects further provide methods of producing stable bendamustine compositions.

As used herein, the term “bendamustine” includes the compound bendamustine, pharmaceutically acceptable salts of bendamustine, isomers, solvates, complexes and hydrates, anhydrous forms thereof, and any polymorphic or amorphous forms or combinations thereof. The salt bendamustine hydrochloride will be discussed herein as a representative of any of these, although the disclosure is not limited to the use of only this salt.

The term “formulation” refers to preparing a drug, e.g., bendamustine, in a form suitable for administration to a patient, such as a human. Thus, a “formulation” can include pharmaceutically acceptable excipients, including diluents or carriers.

The term “lyophilization” refers to processes, in which bendamustine, together with any desired pharmaceutical excipients, is dissolved in a solvent and then subjected to a procedure that involves placing into a dryer and establishing a low shelf temperature, e.g., from −30° C. to 25° C., applying vacuum to obtain a powder residue, and subsequently drying under reduced vacuum to remove residual solvent. Lyophilization processing is suitable for injectables because it can be conducted under sterile conditions, which is a primary requirement for parenteral dosage forms.

The term “dry powder filling” refers to processes including filling a solid particulate composition, such as a mixture of bendamustine and mannitol, into containers such as vials.

As used herein, the term “vial” refers to any walled container, whether rigid or flexible.

“Controlling” as used herein means establishing process conditions to facilitate achievement of a parameter being regulated. For example, in a given case, “controlling” can mean testing samples of each lot or a number of lots regularly or randomly, setting the concentration of degradants as a release specification, choosing process conditions, e.g., use of alcohols and/or other organic solvents in pre-vacuum drying solutions or dispersions, so as to assure that the concentration of degradants of the active ingredient is not unacceptably high, etc. Controlling for degradants by setting release specifications for the amount of degradants can be used to facilitate regulatory approval of a pharmaceutical product by a regulatory agency, such as the U.S. Food and Drug Administration (“FDA”) and similar agencies in other countries or regions.

The term “pharmaceutically acceptable” as used herein describes substances or components that do not cause unacceptable losses of pharmacological activity or unacceptable adverse side effects. Examples of pharmaceutically acceptable ingredients are those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutical composition” as used herein means a composition that is suitable for administration to subjects, including humans, e.g., being made under current good manufacturing procedure conditions and containing pharmaceutically acceptable excipients, e.g., without limitation, any one or more of stabilizers, bulking agents, buffers, carriers, diluents, vehicles, solubilizer, and binders. As used herein, pharmaceutical compositions include, but are not limited to, pre-vacuum dried solutions or dispersions, as well as liquid forms ready for injection or infusion after reconstitution of vacuum dried preparations.

A “pharmaceutical dosage form” as used herein includes pharmaceutical compositions disclosed and in amounts suitable for reconstitution and administration of one or more doses, such as about 1, 1-2, 1-3, 1-4, 1-5, 1-6, 1-10, or about 1-20 doses. Embodiments of a “pharmaceutical dosage form” as used herein include vacuum dried/sterile pharmaceutical compositions disclosed herein in containers and in amounts suitable for reconstitution and delivery of one or more doses, typically about 1, 1-2, 1-3, 1-4, 1-5, 1-6, 1-10, or about 1-20 doses. A pharmaceutical dosage form can comprise a vial or syringe or other suitable pharmaceutically acceptable container. The pharmaceutical dosage forms suitable for injection or infusion use can include sterile aqueous solutions or dispersions or sterile powders, comprising an active ingredient, which are adapted for the preparation of sterile injectable or infusible solutions or dispersions. Generally, the ultimate form for administration should be sterile, fluid, and stable under the conditions of manufacture and storage. A liquid carrier or vehicle can be a solvent or liquid dispersion medium comprising, for example, water, ethanol, and a polyol such as glycerol, propylene glycol, liquid polyethylene glycols, vegetable oils, nontoxic glyceryl esters, and the like and suitable mixtures thereof. The prevention of the growth of microorganisms can be accomplished by incorporating various antibacterial and antimycotic agents, for example, parabens, chlorobutanol, phenol, sorbic acid, thimerosal, and the like.

The term “stable formulation” refers to any preparation of bendamustine having sufficient stability to allow storage at a convenient temperature, such as between about 0° C. and about 60° C., for a commercially reasonable period of time, such as at least about one week, at least about one month, at least about three months, at least about six months, at least about one year, or at least about 2 years.

The term “organic solvent” means an organic material, usually a liquid, that is capable of dissolving other substances. Examples of organic solvents that can be used include, without limitation thereto, acetone, acetonitrile, n-propanol, n-butanol, isopropanol, dimethyl carbonate, dichloromethane, methyl ethyl ketone, methyl isobutyl ketone, 1-pentanol, methyl acetate, methanol, carbon tetrachloride, dimethylsulfoxide, hexafluoroacetone, chlorobutanol, dimethylsulfone, cyclohexane, and any mixtures of two or more thereof. Useful solvents should form stable solutions with bendamustine and not appreciably degrade or deactivate the drug. The solubility of bendamustine in a solvent should be high enough to form commercially useful concentrations of the drug in solvent. Additionally, the solvent should be capable of being removed easily from an aqueous dispersion or solution of the drug product, e.g., through vacuum drying. In embodiments, solutions having concentrations about 2-80 mg/mL, or about 5 to 40 mg/mL, or about 5-20 mg/mL, or about 12 to 17 mg/mL of bendamustine or any of its salt are used.

As used herein, a “trace amount” of an organic solvent means an amount of solvent that is equal to or below recommended levels for pharmaceutical products, for example, as recommended by ICH guidelines (International Conferences on Harmonization, Impurities-Guidelines for Residual Solvents. Q3C, published in the U.S. Federal Register, Vol. 62, No. 247, pages 67377-67388, 1997). The lower limit is the lowest amount that can be detected.

Lyophilization, freeze-drying, or vaccum drying are processes in which solvent is removed from a solution or dispersion after it is frozen and placed under a vacuum, allowing the solvent to change directly from a solid to a vapor without passing through a liquid phase. A process consists of three separate, unique, and interdependent phases: a freezing phase; a primary drying phase (sublimation); and a secondary drying phase (desorption). These processes may be optimized to enhance the product stability as well as decrease the manufacturing costs.

The advantages of lyophilization include ease of processing a liquid, which simplifies aseptic handling; enhanced stability of a dry powder; removal of solvent without excessive heating of the product; enhanced product stability in a dry state; and rapid and easy dissolution of reconstituted product. The product is dried without using elevated temperatures, thereby eliminating adverse thermal effects, and then is stored in the dry state in which there are relatively fewer stability problems. Additionally, freeze dried products are often more soluble, dispersions are stabilized, and products subject to degradation by oxidation or hydrolysis are protected.

Aspects of the present application provide lyophilized bendamustine HCl formulations comprising a bulking agent. As described herein, a lyophilized formulation of bendamustine HCl is achieved following removal of a solvent system from a pre-lyophilization bulk solution. A typical example of solvents used to prepare this formulation includes acetone, acetonitrile, and water for injection, including suitable mixtures of two or more thereof.

A pharmaceutically acceptable excipient can be dissolved in a suitable solvent. Examples of excipients useful for the present application include, without limitation thereto, sodium or potassium phosphate, citric acid, tartaric acid, gelatin, glycine, and carbohydrates such as lactose, sucrose, maltose, glycerin, dextrose, dextran, trehalose and hetastarch. Mannitol is also a useful excipient. Other excipients that may be used, if desired, include antioxidants, such as, without limitation thereto, ascorbic acid, acetylcysteine, cysteine, sodium hydrogen sulfite, butylated hydroxyanisole, butylated hydroxytoluene, alpha-tocopherol acetate, and chelating agents.

In embodiments of the present disclosure, an aqueous pre-vacuum dried solution or dispersion is first formulated. The solution is aseptically filtered into a sterile container, filled into an appropriate sized vial, loosely covered, and loaded into a vacuum dryer. Using vacuum drying techniques, such as those described herein, the solution is dried to obtain moisture contents in the range of about 0.1 to about 8 percent by weight. The resulting dried powder is stable as a powder, typically for about six months to more than about 2 years, or greater than about 3 years, when stored in a closed container at about 5° C. to about 25° C., and can be readily reconstituted with sterile water for injection, or other suitable fluid, to provide liquid formulations of bendamustine, suitable for administration, e.g., by parenteral injection. For intravenous administration, a reconstituted liquid formulation, i.e., a pharmaceutical composition that is a solution, is used.

In specific embodiments, pre-vacuum dried solutions or dispersions can be formulated by: 1) dissolving an excipient, such as mannitol, in water (e.g., up to about 50 mg/mL) at ambient temperature; 2) adding an organic solvent, (about 0.5-99.9% v/v) to the aqueous solution with mixing at about 20-35° C., then optionally reducing the temperature; 4) adding bendamustine hydrochloride to obtain the desired concentration; 5) adding water to achieve a final volume; and 6) subjecting the mixture to vacuum drying in vials. An example of a vacuum drying process involves transferring filled vials into a dryer and establishing a shelf temperature about −30° C., applying a vacuum of less than 500 millitorr for sufficient time to produce a powder residue, subsequently reducing the vacuum to about 300 millitorr, and then to atmospheric pressure, to give the final product.

In another embodiments, pre-vacuum dried solutions or dispersions can be formulated by: 1) dissolving an excipient, such as mannitol, in water (e.g., up to about 60 mg/mL) at ambient temperature and lowering temperature to 2° C.-8° C.; 2) dissolving required quantity of bendamustine HCl in an organic solvent, (about 0.5-99.9% v/v) at a temparature of about −10° C. to 25° C.; 3) mixing both the drug solution and mannitol solution while maintaining a low temparature (−10° C. to 15° C.); 4) adding water to achieve a final volume; and 5) subjecting the mixture to vacuum drying in vials.

Although the preceding steps are shown in a certain order, it is understood that one skilled in the art can change the order of the steps and quantities as desired.

A pre-vacuum dried solution can be sterilized prior to vacuum drying, sterilization sometimes being performed by aseptic filtration, e.g., through a 0.22 μm or smaller pore filter. Multiple sterilization filters can be used. Sterilization of the solution or dispersion can be achieved using other methods known in the art, e.g., by exposure to radiation.

After sterilization, the solution is subjected to vacuum drying. Generally, the filtered solution will be introduced into a sterile receiving vessel, and then transferred to any suitable container or containers in which the formulation may be effectively dried under vacuum. In embodiments, the formulation is effectively and efficiently dried in the containers in which the product is to be marketed, such as, without limitation, a vial, as described herein and as known in the art.

An example of a procedure for use in vacuum drying the pre-vacuum dried solutions or dispersions is set forth below. However, a person skilled in the art would understand that modifications to the procedure or process might be made, depending on characteristics of the pre-vacuum dried solution or dispersion and the lyophilization equipment.

Once the drying cycle is completed, the vacuum in the chamber can be slowly released to atmospheric pressure (or slightly below), such as by introducing sterile, dry nitrogen gas or another inert gas. If the product composition has been vacuum dried in containers such as vials, the vials can then be completely stoppered and sealed.

The formulations of present disclosure can be dry powders that are filled into vials, referring to a process where a drug is directly obtained aseptically, mixed with excipients such as sterile bulking agents, and then filled into vials.

In embodiments, the present application utilizes processes of lyophilization for bendamustine, using mixtures of at least acetonitrile and water, or acetonitrile alone, or organic solvent alone, or mixtures of acetonitrile with other organic solvents.

In embodiments, the present application utilizes processes of lyophilization for bendamustine, using mixtures of at least acetone and water, or acetone alone, or organic solvent alone, or mixtures of acetone with other organic solvents.

As described herein, a lyophilized formulation of bendamustine is achieved following removal of an organic solvent and water. In embodiments, a solvent used to prepare a formulation comprises acetone or acetonitrile. Other organic solvents can be used, including, but not limited to, n-propanol, n-butanol, isoproponal, dimethyl carbonate, tertiary-butanol (TBA), N,N-dimethylacetamide, dichloromethane, methyl ethyl ketone, methyl isobutyl ketone, 1-pentanol, methyl acetate, methanol, carbon tetrachloride, dimethylsulfoxide, hexafluoroacetone, chlorobutanol, dimethylsulfone, and cyclohexane. These solvents may be used individually or in any combinations of two or more. Useful solvents will form stable solutions with bendamustine and not appreciably degrade or deactivate the drug. The solubility of bendamustine in the selected solvent should be high enough to form commercially useful concentrations of the drug. Additionally, the solvent should be capable of being removed easily from an aqueous dispersion or solution of the drug product, e.g., through lyophilization.

The process of lyophilization includes three separate, unique, and interdependent phases: a freezing phase: a primary drying phase (sublimation) and a secondary drying phase (desorption). A primary function of the freezing phase is to ensure that the entire container having the complex solution is completely frozen, prior to proceeding to a subsequent phase. Additionally, it is usually desired that of the containers in a batch freeze in a uniform manner. While there are different ways that this can be accomplished, one option is chilling the containers after they are loaded onto the lyophilizer shelves and holding (such as for 30-60 minutes) prior to initiation of the freezing cycle. It is generally not practical to equilibrate the shelves to a freezing temperature, because of frost accumulation during the filling and loading of the containers.

Once the formulation is brought to the desired frozen state, primary drying by sublimation can proceed. The primary drying phase involves the removal of bulk water at a product temperature below the ice transition temperature under a vacuum (e.g., at pressures about 50-300 mTorr). The goal is to identify the glass transition temperature (Tg′) for the formulation. The Tg′ is the temperature at which there is a reversible change of state between a viscous liquid and a rigid, amorphous glassy state. One can measure the Tg′ of candidate formulations using differential scanning calorimetry (DSC), in particular with modulated DSC. Generally, the collapse temperature is observed to be about 2-5° C. greater than the Tg′. Hence, the shelf temperature is set such that the target product temperature is maintained near or below the Tg′ of the formulation throughout the removal of solvent during the primary drying phase.

As the solvent is progressively removed from the formulated containers, the product temperature will approach and reach the shelf temperature, since it is no longer cooled by water sublimation. To optimize the duration of the primary dry phase, the removal of solvent vapor can be tracked using a moisture detector, or by monitoring the decrease in pressure difference between a capacitance manometer and a thermocouple pressure gauge, or by a pressure drop measurement. The optimization of the primary dry cycle involves a removal of solvent as quickly as possible without causing cake collapse and subsequent product instability.

A secondary drying phase is the final segment of the lyophilization cycle, where residual moisture is removed from a formulation's interstitial matrix by desorption with elevated temperatures and/or reduced pressures. The final moisture content of a lyophilized formulation, which can be measured by a Karl Fisher technique or other methods, is important because if the cake contains too much residual moisture, the stability of the active can be compromised. Hence, it is desired to achieve a moisture level as low as possible.

To attain a low residual moisture, the shelf temperature is typically elevated to accelerate desorption of water molecules. The duration of the secondary drying phase is usually short. When microstructure collapse occurs, the residual moisture is generally significantly greater than desired. One alternative is to purge the sample chamber of the lyophilizer with alternating cycles of an inert gas, such as nitrogen, to facilitate displacement of bound water.

For example, a lyophilization process can include the following steps:

1) A solvent is maintained at temperatures about −30° C. to −10° C.

2) One or more suitable excipients is added to the solvent

3) Drug is added to the step 2) mixture with stirring until it completely dissolves.

4) The volume is made up to a desired quantity with solvent at temperatures about −30° C. to −10° C.

5) The solution is filtered through a sterilizing filter, filled into a container, and the container is loosely covered

7) Covered containers are loaded into a lyophilizer with pre-cooled shelves

8) The solution in the containers is lyophilized.

Pharmaceuticals to be freeze-dried are frequently in aqueous solutions, ranging from about 0.01 to 40% by weight concentrations of total solids. Usually, an improvement in stability of the lyophilisate, compared to a solution, results from a substantial absence of water in the lyophilizate.

The pharmaceutical dosage form products of the present disclosure, although typically in vial packaging, may be any suitable container, such as ampoules, syringes, or co-vials, which are capable of maintaining a sterile environment. Such containers can be made of glass or plastic, provided that the material does not interact with the bendamustine formulation. The closures can be stoppers, such as a sterile rubber stoppers, in instances bromobutyl rubber stoppers, which afford a hermetic seal.

In embodiments, a vial will contain a powder including about 10-500 mg of bendamustine or a salt thereof, and about 5 mg to 2 g of a sugar or sugar alcohol. In embodiments, a vial will contain about 25-100 mg of bendamustine or a salt thereof, and about 10-300 mg of a sugar or sugar alcohol. In specific embodiments, the sugar alcohol comprises mannitol.

The formulations of the present disclosure may be reconstituted with water, such as sterile water for injection, or another sterile fluid such as a co-solvent, to provide an appropriate solution of bendamustine for administration, as through parenteral injection following further dilution into an appropriate intravenous fluid, for example, normal saline.

A final dilution of the reconstituted bendamustine in a formulation of the application may be carried out with other preparations having similar utility, for example, 5% dextrose injection, lactated Ringer's and dextrose injection, sterile water for injection, and the like. In embodiments, because of its narrow pH range, pH 6 to 7.5, lactated Ringer's Injection is used; Lactated Ringer's injection contains NaCl 0.6 g, sodium lactate 0.31 g, KCl 0.03 g, and CaCl₂.2H₂O 0.02 g, per 100 mL. The osmolarity is 275 mOsmol/L, which is very close to isotonicity.

Lyophilized preparations, and reconstituted solutions, can contain small amounts of “bendamustine-related” impurities that are the result of decomposition or degradation of bendamustine and its salts during pharmaceutical product manufacturing and storage, or are artifacts from processes for synthesizing the drug.

In embodiments, the present application provides lyophilized bendamustine formulations wherein the content of HP-1 impurity is not greater than about 0.5%. In embodiments, the content of HP-1 Ester impurity is not greater than about 0.2%. In embodiments, the content of HP-2 impurity is not greater than about 0.2%. In embodiments, the content of Methyl Ester impurity is not greater than about 0.2%. In embodiments, the content of Ethyl Ester impurity is not greater than about 0.2%. In embodiments, the content of the BEN-2 impurity is not greater than about 0.2%. In embodiments, the content of the highest individual unidentified bendamustine-related impurity is not greater than about 0.7%. In embodiments, the content of total bendamustine-related impurities is not greater than about 0.6%. All of the impurity contents herein are expressed as percentages of the label drug content. Solutions of bendamustine can be analyzed by common techniques, such as high performance liquid chromatograpy, to determine their bendamustine content and the concentrations of bendamustine-related impurities.

The identities of the specific impurities mentioned above are as shown below.

| Impurity | Chemical Name | Structure |
|---|---|---|
| HP-1 | 4-{5-[(2-Chloro-ethyl)-(2- hydroxy-ethyl)-amino]-1- methyl-1H- benzoimidazol-2-yl}- butyric acid | 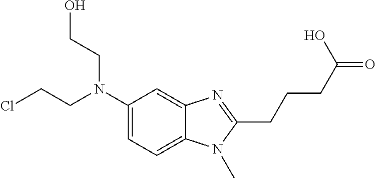 [SMILES: Cn1c(CCCC(=O)O)nc2cc(N(CCO)CCCl)ccc21] |
| HP-1 Ester | 4-{5-[(2-Chloro-ethyl)-(2- hydroxy-ethyl)-amino]-1- methyl-1H- benzoimidazol-2-yl}- butyric acid isopropyl ester | 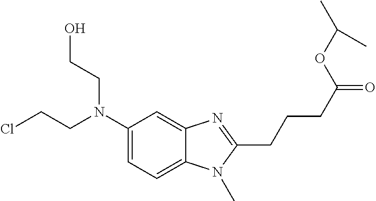 [SMILES: CC(C)OC(=O)CCCc1nc2cc(N(CCO)CCCl)ccc2n1C] |
| HP-2 | 4-{5-[Bis-(2-hydroxy- ethyl)-amino]-1-methyl- 1H-benzoimidazol-2-yl}- butyric acid | 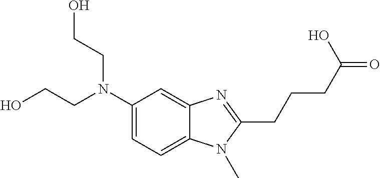 [SMILES: Cn1c(CCCC(=O)O)nc2cc(N(CCO)CCO)ccc21] |
| Methyl Ester | 4-{5-[Bis-(2-chloro-ethyl)- amino]-1-methyl-1H- benzoimidazol-2-yl}- butyric acid methyl ester | 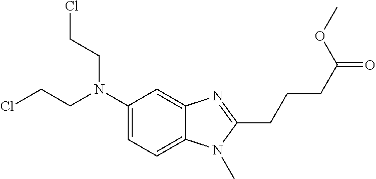 [SMILES: COC(=O)CCCc1nc2cc(N(CCCl)CCCl)ccc2n1C] |
| Ethyl Ester | 4-{5-[Bis-(2-chloro-ethyl)- amino]-1-methyl-1H- benzoimidazol-2-yl}- butyric acid ethyl ester | 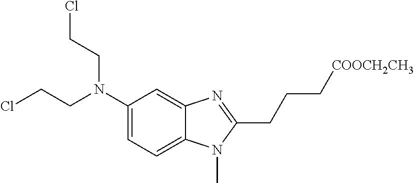 |
| BEN-2 | 4-{5-[Bis-(2-chloro-ethyl)- amino]-1-methyl-1H- benzoimidazol-2-yl}- butyric acid isopropyl ester | 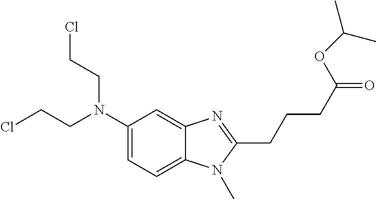 [SMILES: CC(C)OC(=O)CCCc1nc2cc(N(CCCl)CCCl)ccc2n1C] |

The following examples will further describe certain specific aspects and embodiments of the disclosure, are being provided solely for purposes of illustration, and the disclosure should not be considered as being limited thereto.

### EXAMPLES 1-6

| | Milligrams per Unit | | | | | |
|---|---|---|---|---|---|---|
| Ingredient | 1 | 2 | 3 | 4 | 5 | 6 |
| Bendamustine HCl | 100 | 100 | 100 | 100 | 100 | 100 |
| Mannitol | 170 | — | — | 170 | 200 | — |
| Sorbitol | — | 170 | — | — | — | 200 |
| Sucrose | — | — | 170 | — | — | — |

Solvents (evaporate during processing)

| | | | | | | |
|---|---|---|---|---|---|---|
| t-Butanol | q.s. | — | — | q.s. | — | — |
| Water | q.s. | q.s. | q.s. | — | q.s. | q.s. |
| DMSO | — | — | — | — | q.s. | — |
| Acetonitrile | — | q.s. | — | — | — | — |
| Acetone | — | — | q.s. | — | — | — |

Manufacturing procedure: the required ingredients are mixed to form a solution, and then the solution is processed using vacuum drying or lyophilization to form a solid. Appropriate quantities of the solid are contained in vials.

### EXAMPLE 7

| | Quantity per Unit | | | |
|---|---|---|---|---|
| Ingredient | 7A | | 7B | |
| Bendamustine HCl | 25 | mg | 100 | mg |
| Mannitol | 42.5 | mg | 170 | mg |
| Acetone* | 0.3125 | mL | 1.25 | mL |

| | | |
|---|---|---|
| Water for Injection* | q.s. to 1.125 mL | q.s. to 4.5 mL |

*Evaporates during processing.

Manufacturing procedure:

1. Bendamustine HCl is mixed with acetone.

2. The suspension temperature is lowered to 0° C. to −10° C.

3. About 30% of the water for injection (at 2-25° C.) is added to the cooled suspension.

4. The temperature is lowered to 0° C. to −7° C. with continuous stirring.

5. Mannitol is dissolved in about 55% of the water for injection.

6. Mannitol solution is added to the material of step 4 with continuous stirring, and the temperature is lowered to 0° C. to −7° C.

7. Remaining water for injection is added and the mixture is stirred while maintaining the temperature between 0° C. and −7° C. under a nitrogen atmosphere.

8. The solution of step 7 is filtered in two steps through 0.22 μm PVDF sterile filters.

9. Appropriate amounts of filtered solution from step 8 is filled into depyrogenated vials (tubular type I glass vials) and the vials are loosely covered with slotted sterile chlorobutyl rubber stoppers. The temperature of the solution is maintained between 0° C. and −7° C. during filling.

10. Covered vials are loaded onto lyophilizer pre-cooled shelves maintained at −35° C. The vials are loaded immediately after filling and if any vial is not loaded in the lyophilizer within 20 minutes after filling, then the vial is discarded.

11. The vials are lyophilized in a freeze dryer, with the following parameters.

| | | Vacuum | Time |
|---|---|---|---|
| Step | Temperature | (mBar) | (minutes) |
| Freezing | −35° C. to −48° C. | — | 1210 |
| Primary drying | −48° C. to 32° C. | 200-0.07 | 3210 |
| Secondary drying | 32° C. | 0.07 | 200 |

12. After completion of lyophilization, the vacuum is reduced by flushing with sterile nitrogen, the vials are stoppered completely, and the vials are sealed with flip-off seals.

Vials are stored at 25±2° C. and 60% relative humidity (RH), or at 40±2° C. and 75% RH, for 1, 2, and 3 month periods. Stored vials are used to determine drug stability parameters. A determination of pH is performed after mixing the contents of a vial and water.

To determine impurity contents and the drug assay, the contents of a vial is dissolved using a diluent (potassium dihydrogen orthophosphate, water and acetonitrile) and the drug content or individual impurity content is measured using a HPLC analytical method.

Results are shown in the following tables, where drug assay and impurity values are expressed as percentages of the label drug content, and comparison results for two batch numbers of the corresponding commercial TREANDA product and the products as initially prepared above are also shown in each table.

| | | | Example 7A | | | | | | |
|---|---|---|---|---|---|---|---|---|---|
| | TREANDA† | | | 25° C. and 60% RH | | | 40° C. and 75% RH | | |
| Test | A | B | Initial | 1 Mo. | 2 Mo. | 3 Mo. | 1 Mo. | 2 Mo. | 3 Mo. |
| pH | — | 2.98 | 2.98 | 2.98 | 3.00 | 3.06 | 3.00 | 3.05 | 3.07 |
| Drug | 103.9 | 102.7 | 108.0 | 107.1 | 107.5 | 106.7 | 107.1 | 107.8 | 107.0 |
| Assay | | | | | | | | | |

Drug-Related Impurities

| | | | | | | | | | |
|---|---|---|---|---|---|---|---|---|---|
| HP-1 | 0.28 | 0.23 | 0.10 | 0.09 | 0.16 | 0.17 | 0.12 | 0.16 | 0.14 |
| HP-1 | ND | ND | ND | ND | ND | ND | ND | ND | ND |
| Ester | | | | | | | | | |
| HP-2 | ND | ND | ND | ND | ND | ND | ND | ND | ND |
| Methyl | ND | ND | ND | ND | ND | ND | ND | ND | ND |
| Ester | | | | | | | | | |
| Ethyl | ND | ND | ND | ND | ND | ND | ND | ND | ND |
| Ester | | | | | | | | | |
| BEN-2 | ND | ND | ND | ND | 0.02 | 0.02 | ND | 0.02 | 0.02 |
| HUI* | 0.13 | 0.13 | 0.02 | 0.03 | 0.04 | 0.04 | 0.08 | 0.08 | 0.06 |
| Total | 0.77 | 0.75 | 0.14 | 0.15 | 0.25 | 0.27 | 0.24 | 0.36 | 0.32 |

ND = not detected.
*HUI = highest unidentified impurity.
†A = Batch No. TB30410 and B = Batch No. TB30310.

| | | | Example 7B | | | | | | |
|---|---|---|---|---|---|---|---|---|---|
| | TREANDA† | | | 25° C. and 60% RH | | | 40° C. and 75% RH | | |
| Test | A | B | Initial | 1 Mo. | 2 Mo. | 3 Mo. | 1 Mo. | 2 Mo. | 3 Mo. |
| pH | — | 2.95 | 2.95 | 3.01 | 3.06 | 3.07 | 3.00 | 3.03 | 3.08 |
| Drug | 103.7 | 102.8 | 98.7 | 99.1 | 100.7 | 101.6 | 98.8 | 100.8 | 101.2 |
| Assay | | | | | | | | | |

Drug-Related Impurities

| | | | | | | | | | |
|---|---|---|---|---|---|---|---|---|---|
| HP-1 | 0.26 | 0.36 | 0.12 | 0.11 | 0.15 | 0.24 | 0.14 | 0.19 | 0.24 |
| HP-1 | ND | ND | ND | ND | ND | ND | ND | ND | ND |
| Ester | | | | | | | | | |
| HP-2 | ND | ND | ND | ND | ND | ND | ND | ND | ND |
| Methyl | ND | ND | ND | ND | ND | ND | ND | ND | ND |
| Ester | | | | | | | | | |
| Ethyl | ND | ND | ND | ND | ND | ND | ND | ND | ND |
| Ester | | | | | | | | | |
| BEN-2 | ND | ND | ND | 0.01 | 0.02 | 0.02 | 0.02 | 0.02 | 0.02 |
| HUI* | 0.21 | 0.23 | 0.04 | 0.02 | 0.04 | 0.05 | 0.03 | 0.07 | 0.10 |
| Total | 0.85 | 0.90 | 0.19 | 0.18 | 0.21 | 0.38 | 0.18 | 0.41 | 0.51 |

ND = not detected.
*HUI = highest unidentified impurity.
†A = Batch No. TA31810 and B = Batch No. TA32310.

A test to determine the time required for reconstitution of lyophilized material in the vials of Example 7A is conducted by mixing the contents of a vial with 5 mL of water for injection, and the times required to form a visibly clear solution are noted. Results are shown in the following table.

| Storage Condition | | Time (seconds) |
|---|---|---|
| Initial | | 55 |
| 25° C. and | 1 Month | 53 |
| 60% RH | 2 Months | 50 |
| | 3 Months | 48 |
| 40° C. and | 1 Month | 50 |
| 75% RH | 2 Months | 48 |
| | 3 Months | 50 |

Reproducibility of reconstitution times for eight batches prepared as described for Example 7B is determined, using the procedure described above but with 20 mL of water for injection. Results are shown in the following table.

| Batch No. | Time (seconds) |
|---|---|
| BEI1-041 | 55 |
| BEI1-045A | 53 |
| BEI1-045B | 50 |
| BEI1-045C | 50 |
| BEI1-051A | 48 |
| BEI1-051B | 48 |
| BEI1-051C | 50 |
| BEI1-051D | 50 |

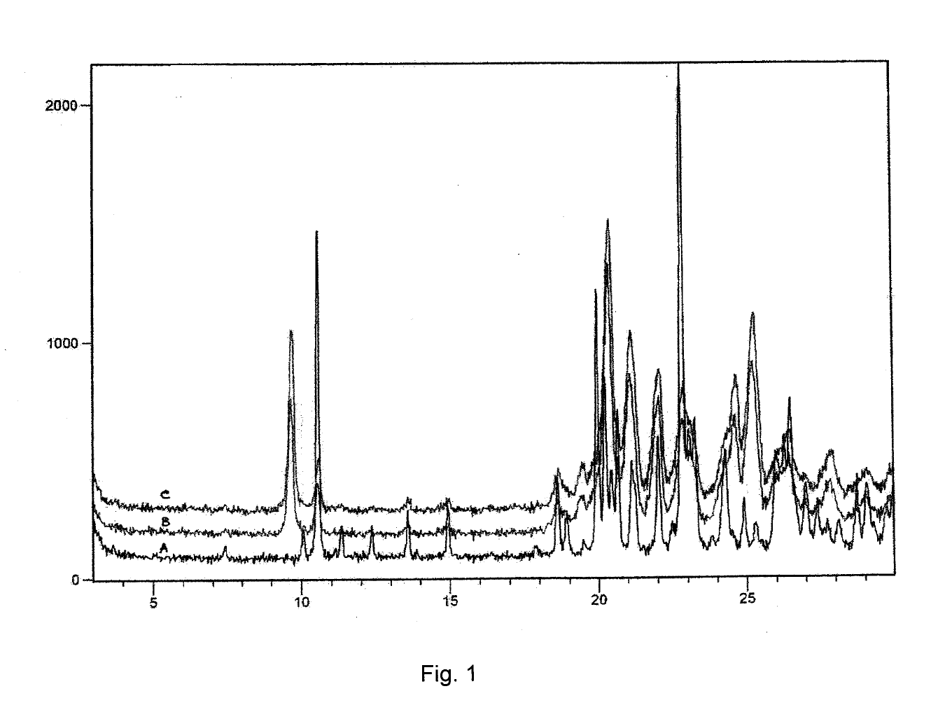

FIG. 1 shows PXRD patterns of: the bendamustine HCl ingredient (A); a lyophilized product prepared according to Example 7A (B); and a lyophilized product prepared according to Example 7B (C).

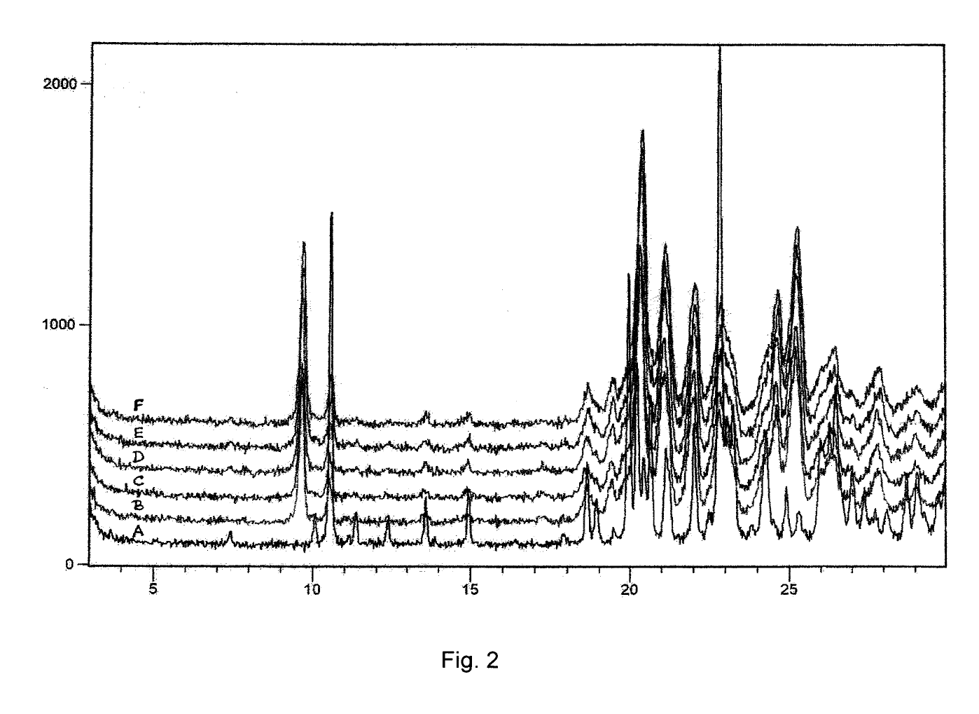

FIG. 2 shows PXRD patterns of: the bendamustine HCl ingredient (A); a lyophilized product prepared according to Example 7B (F); a lyophilized product prepared according to Example 7B, after storage at 25±2° C. and 60% RH for 1 month (E); a lyophilized product prepared according to Example 7B, after storage at 25±2° C. and 60% RH for 2 months (D); a lyophilized product prepared according to Example 7B, after storage at 40±2° C. and 75% RH for 1 month (C); and a lyophilized product prepared according to Example 7B, after storage at 40±2° C. and 75% RH for 2 months (B).

These PXRD patterns indicate the drug polymorphic stability in the lyophilized preparations, and are obtained using copper Kα radiation. In the figures, the y-axis is intensity units and the x-axis is the 2θ angle, in degrees.

### EXAMPLE 8

| Ingredient | Quantity per Unit | | | |
|---|---|---|---|---|
| Bendamustine HCl | 25 | mg | 100 | mg |
| Mannitol | 42.5 | mg | 170 | mg |
| Acetonitrile* | 0.3125 | mL | 1.25 | mL |

| | | |
|---|---|---|
| Water for Injection* | q.s. to 1.125 mL | q.s. to 4.5 mL |

* Evaporates during processing.

Manufacturing procedure: similar to the procedure of Example 7, except that acetonitrile is used in place of acetone to prepare pre-lyophilization solution.

### EXAMPLE 9

| Ingredient | Quantity per Unit | | | |
|---|---|---|---|---|
| Bendamustine HCl | 25 | mg | 100 | mg |
| Mannitol | 30 | mg | 120 | mg |
| Acetone* | 0.3125 | mL | 1.25 | mL |

| | | |
|---|---|---|
| Water for Injection* | q.s. to 1.125 mL | q.s. to 4.5 mL |

* Evaporates during processing.

Manufacturing procedure: similar to the procedure of Example 7.

1. A lyophilized pharmaceutical formulation of bendamustine, comprising not more than about 0.4% by weight, based on the bendamustine content, of a compound having the structure: 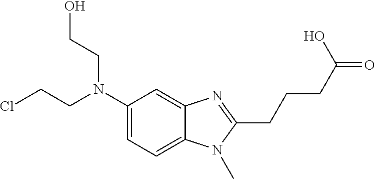 [SMILES: Cn1c(CCCC(=O)O)nc2cc(N(CCO)CCCl)ccc21]
the formulation being prepared by lyophilizing a solution that does not contain tertiary-butyl alcohol or ethanol.

2. The lyophilized pharmaceutical formulation according to claim 1, being prepared by lyophilizing a solution containing water, at least one organic solvent, or a mixture thereof

3. The lyophilized pharmaceutical formulation according to claim 1, being prepared by lyophilizing a solution containing an organic solvent that is acetone, acetonitrile, n-propanol, n-butanol, isopropanol, methanol, ethyl acetate, dimethyl carbonate, dichloromethane, methyl ethyl ketone, methyl isobutyl ketone, 1-pentanol, methyl acetate, carbon tetrachloride, dimethylsulfoxide, hexafluoroacetone, chlorobutanol, dimethylsulfone, acetic acid, cyclohexane, or a mixture of two or more thereof.

4. The lyophilized pharmaceutical formulation according to claim 1, 2 or 3, being prepared by lyophilizing a solution having a solvent that is water and 5 to 50% by volume of an organic solvent.

5. The lyophilized pharmaceutical formulation according to claim 1, containing a bulking agent.

6. The lyophilized pharmaceutical formulation according to claim 1, containing a bulking agent that is mannitol, lactose, sucrose, or any mixtures thereof

7. The lyophilized pharmaceutical formulation according to claim 1, containing a bulking agent that is mannitol.

8. The lyophilized pharmaceutical formulation according to claim 7, comprising about 25-100 mg of bendamustine or a salt thereof and about 10 mg to about 300 mg of mannitol, present in a single-use container.

9. The lyophilized pharmaceutical formulation of bendamustine according to claim 1, being prepared by lyophilizing a solution having a solvent comprising water and acetone.

10. The lyophilized pharmaceutical formulation of bendamustine according to claim 1, being prepared by lyophilizing a solution having a solvent comprising water and acetonitrile.

11. A solution or dispersion for lyophilization, comprising bendamustine hydrochloride, mannitol, water, and an organic solvent, wherein an organic solvent does not include tertiary-butyl alcohol or ethanol.

12. The solution or dispersion for lyophilization according to claim 11, comprising a solvent that is water and 5 to 50% by volume of an organic solvent.

13. The solution or dispersion for lyophilization according to claim 11, comprising a solvent that is water and 5 to 50% by volume of acetone, acetonitrile, or a mixture thereof.

14. The lyophilized pharmaceutical formulation according to claim 4, having bendamustine-related impurities totaling less than 0.7% of the label bendamustine content.

15. The lyophilized pharmaceutical formulation according to claim 4, having bendamustine-related impurities totaling less than 0.6% of the label bendamustine content.

16. The lyophilized pharmaceutical formulation of claim 4, further comprising mannitol and having a reconstitution time with an aqueous fluid that is less than 120 seconds.

17. The lyophilized pharmaceutical formulation of claim 4, further comprising mannitol and having a reconstitution time with an aqueous fluid that is less than 60 seconds.
